(19) United States
(12) Patent Application Publication
(10) Pub. No.: US 2026/0263702 A1
(43) Pub. Date: Sep. 10, 2026

(54) A needle cover and a subassembly of a medicament delivery device

(71) Applicant: SHL Medical AG, Zug (CH)

(72) Inventors: Daniel Säll, Nacka Strand (SE); Daniel Carlsson, Nacka Strand (SE)

(21) Appl. No.: 18/875,797

(22) PCT Filed: Jun. 9, 2023
(86) PCT No.: PCT/EP2023/065471

§ 371 Date: Dec. 17, 2024

(30) Foreign Application Priority Data
Jun. 23, 2022 (EP) 22180600.3

(51) Int. Cl. A61M 5/32; A61M 5/20; A61M 5/31

(52) U.S. Cl. CPC A61M 5/3202; A61M 5/2033; A61M 5/3129; A61M 2005/3142

(57) ABSTRACT

The present disclosure generally relates to medicament delivery devices such as autoinjectors, and particularly concerns a needle cover and a subassembly for removing a needle shield.

### CROSS-REFERENCE TO RELATED APPLICATIONS

The present application is a U.S. National Phase Application pursuant to 35 U.S.C. § 371 of International Application No. PCT/EP2023/065471 filed Jun. 9, 2023, which claims priority to EP Patent Application No. 22180600.3 filed Jun. 23, 2022. The entire disclosure contents of these applications are herewith incorporated by reference into the present application.

### TECHNICAL FIELD

The present disclosure generally relates to medicament delivery devices such as autoinjectors, and particularly concerns a needle cover and subassembly for removing a needle shield.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning removal of a rigid needle shield of the medicament delivery devices, which are set out in more detail below.

### SUMMARY

An object of the present disclosure is to provide a needle cover and a subassembly for a medicament delivery device which solves, or at least mitigates problems of the prior art.

According to a first aspect of the present disclosure, there is provided a needle cover configured to cover a needle of a medicament delivery device, the needle being attached to a proximal end of a medicament container, the needle cover comprising: a proximal tubular portion defining a proximal opening through which a needle extends in a proximal direction during an injection event, at least one protrusion extending radially outwards and being configured to slide against a surface of a housing accommodating the needle cover, wherein at least one of the protrusion and the surface of the housing includes a curved surface to cause the needle cover to move proximally with respect to the housing when the needle cover is rotated axially relative the housing; and at least one protrusion arranged inside the proximal tubular portion, the at least one protrusion extends radially inwards towards the longitudinal axis of the needle cover, the at least one protrusion extending radially inwards is configured to engage with a distally facing surface of a removable shield member arranged to cover the needle, and to push the removable shield member in the proximal direction to remove the removable shield member from the medicament container.

Embodiments of the present disclosure advantageously provides for removal of the removable shield member when the needle cover is moved in the proximal direction. In this way, the needle is firstly protected by the removable needle shield, and once the needle cover is moved in the proximal direction by the interaction with the surface of the housing the needle cover push the removable shield member so that it is removed from the medicament container and the needle is exposed. However, the proximal motion of the needle cover ensures that the needle is covered until the medicament delivery device is in use and the needle cover is moved distally.

The interface between the protrusion of the needle cover and the surface of the housing may be considered a cam interface that causes proximal motion of the needle cover when it is rotated with respect to the housing.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction 31 relative to an axis 112, typically a central axis extending in the direction of the longest extension of the device and/or component. Similarly, “radial” or “radially” refer to a direction 32 extending radially relative to the axis, and “rotation”, “rotational” and “rotationally” refer to rotation relative to the axis.

According to one embodiment, the at least one protrusion extending radially inwards may an annular flange configured to abut against and push on a distally facing surface of the removable shield member. A flange provides for evenly distributed force to be applied on the removable shield member for reliable removal.

According to one embodiment, the at least one protrusion extending radially inwards may be a set of flexible tabs annularly arranged around an inner circumference of the needle cover and being configured to abut against and push on a distally facing surface of the removable shield member. A set of tabs provides for evenly distributed force to be applied on the removable shield member for reliable removal thereof.

According to one embodiment, the proximal tubular portion is configured to shield the needle after an injection event is completed. This advantageously reduces the risk of sharp injury after the injection event.

According to one embodiment, the at least one protrusion extending radially inwards may be arranged at the distal end of the proximal tubular portion. This facilitates for the protrusion to reach and contact the shield member.

According to one embodiment, the needle cover may comprise two protrusions extending radially outwards symmetrically arranged on the needle cover on opposite sides of the longitudinal axis. This advantageously provides for more reliable proximal motion of the needle cover when the needle cover is rotated axially relative the housing.

According to a second aspect of the present disclosure, there is provided a subassembly of a medicament delivery device, the subassembly comprising: a housing extending in a longitudinal direction from a proximal end to a distal end relative the longitudinal axis, a needle cover arranged in the housing and configured to cover a needle at a proximal end of the housing, the needle cover is axially movable in relation to the housing along a longitudinal axis between a distal position and a proximal position; a removable shield member configured to cover a needle, the needle being attached to a proximal end of a medicament container accommodated in the housing; the needle cover comprising at least one protrusion extending radially outwards and being configured to slide against a surface of the housing, wherein at least one of the protrusion and the surface of the housing includes a curved surface to cause the needle cover to move proximally with respect to the needle when the needle cover is rotated axially relative the housing; and the needle cover further comprising at least one protrusion extending radially inwards towards a longitudinal axis of the needle cover, the least one protrusion extending radially inwards is adjacent to a distally facing surface of the removable shield member.

The least one protrusion extending radially inwards is adjacent to the distally facing surface of the removable shield member to remove the removable shield member from the medicament container when the needle cover moves in a proximal direction.

According to one embodiment, the subassembly may comprise a proximal housing part removably attachable to the housing of the medicament delivery device at a proximal end of the housing.

According to one embodiment, the proximal housing part comprises a slot extending parallel with the longitudinal axis in which the at least one protrusion extending radially outwards of the needle cover is arranged. Advantageously, the proximal housing part being axially rotatable with respect to the housing, wherein the needle cover is rotated by the interaction between the slot and the at least one protrusion extending radially outwards when the proximal housing part is rotated towards a locked position in the housing.

According to one embodiment, the at least one protrusion extending radially outwards of the needle cover may advantageously abut against a distally facing surface of the housing to lock the proximal housing part in a locked position.

According to one embodiment, the needle cover may initially be in a distal position in which the removable shield member is exposed at the proximal end of the proximal housing part.

According to one embodiment, the proximal motion of the needle cover caused by the interaction with the surface of the housing may move the needle cover to a proximal position relative the distal position.

According to one embodiment, the at least one protrusion extending radially inwards may be an annular flange adjacent to the distally facing surface of the removable shield member.

According to one embodiment, the at least one protrusion extending radially inwards may be a set of flexible tabs annularly arranged around an inner circumference of the needle cover and being adjacent to the distally facing surface of the removable shield member.

According to one embodiment, the surface of the housing may comprise the curved surface.

According to one embodiment, the at least one protrusion of the needle cover extending radially outwards may comprise the curved surface configured to slide against the surface that is part of a protrusion of the housing.

Further embodiments of, and effects obtained through this second aspect of the present disclosure are largely analogous to those described above for the first aspect of the present disclosure.

There is further provided a medicament delivery device comprising the subassembly of any of the herein disclosed embodiments.

According to a third aspect of the present disclosure there is provided a medicament delivery device comprising: a proximal disposable part arranged with a proximal housing and a needle cover telescopically arranged in relation to the proximal housing; wherein the proximal disposable part (201) is configured to receive a medicament container; a distal reusable part detachably arranged with the proximal disposable part; wherein the needle cover has an extension position in which the needle cover is extended in the proximal direction to cover a needle of the medicament container, and a distal position in which the needle cover is retracted in the distal direction to expose the needle; wherein the distal reusable part comprises a plunger unit and a plunger rod spring; wherein the plunger unit is configured to move a stopper of the medicament container in the proximal direction upon being moved by the plunger rod spring, so that a medicament contained in the medicament container is expelled; wherein the distal reusable power pack comprises a housing, the housing being configured to receive the proximal disposable part.

According to one embodiment, medicament delivery device may further comprise a locking element configured to prevent the plunger rod from moving axially in relation to the housing in a locking position.

medicament delivery device, the needle cover may comprise a trigger; wherein the trigger is configured to release the locking element to move out from the locking position, thereby releasing the plunger unit to move in the proximal direction.

According to one embodiment, medicament delivery device may comprise an activation interface that is movable in the housing, wherein the trigger of the needle cover contacts the locking element via the activation interface to release the locking element to move out from the locking position.

According to one embodiment, the needle cover may comprise a distally extending arm configured to contact the locking element to release the locking element to move out from the locking position.

According to one embodiment, the locking element may be a locking ring that is transversely movable in the housing to move out from the locking position.

According to one embodiment, the needle cover may comprise: at least one protrusion extending radially outwards and being configured to slide against a surface of the housing to cause the needle cover to move proximally with respect to the needle when the needle cover is rotated axially relative the housing once received in the housing; and at least one protrusion extending radially inwards towards a longitudinal axis of the needle cover, the least one protrusion extending radially inwards is adjacent to a distally facing surface of a removable shield member covering the needle.

According to one embodiment, the proximal disposable part may be configured to be rotated with respect to reusable part for attachment with the reusable part whereby the rotation causes the rotation of the needle cover axially relative the housing, wherein the proximal motion of the needle cover causes the at least one protrusion extending radially inwards to remove the removable shield member from the medicament container.

According to one embodiment, the rotation of the needle cover may cause the trigger to be is aligned with the activation interface.

According to one embodiment, the rotation of the needle cover may cause the distally extending arm of the needle cover to be is aligned with the locking element.

According to one embodiment, the surface of the housing may comprise the curved surface.

According to one embodiment, the at least one protrusion of the needle cover extending radially outwards may comprise the curved surface configured to slide against the surface that is part of a protrusion of the housing.

According to one embodiment, the proximal housing part may comprise a slot extending parallel with the longitudinal axis in which the at least one protrusion extending radially outwards of the needle cover is arranged.

According to one embodiment, the at least one protrusion extending radially outwards of the needle cover may abut against a distally facing surface of the housing to lock the proximal housing part in a locked position.

According to one embodiment, the needle cover may initially be in a distal position in which the removable shield member is exposed at the proximal end of the proximal housing part.

According to one embodiment, the proximal motion of the needle cover caused by the interaction with the surface of the housing may move the needle cover to a proximal position relative the distal position.

According to one embodiment, the at least one protrusion extending radially inwards may be an annular flange adjacent to the distally facing surface of the removable shield member.

According to one embodiment, the at least one protrusion extending radially inwards may be a set of flexible tabs annularly arranged or disposed around an inner circumference of the needle cover and being adjacent to the distally facing surface of the removable shield member.

Further embodiments of, and effects obtained through this third aspect of the present disclosure are largely analogous to those described above for the first aspect and the second aspect of the present disclosure.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an autoinjector according to embodiments of the present disclosure;

FIG. 2 is an exploded view of an autoinjector according to embodiments of the present disclosure;

FIG. 3 is a perspective view of a needle cover according to embodiments of the present disclosure;

FIG. 4 is another perspective view of the needle cover according to embodiments of the present disclosure;

FIG. 5 is yet another perspective view of the needle cover according to embodiments of the present disclosure;

FIG. 6 is a perspective view of a medicament container and a removable needle shield according to embodiments of the present disclosure;

FIG. 7 is a perspective view of a medicament container and a removable needle shield according to embodiments of the present disclosure;

FIG. 8 is a perspective view of a housing according to embodiments of the present disclosure;

FIG. 9 is an exploded view of a subassembly according to embodiments of the present disclosure;

FIG. 10 is a perspective view of a proximal housing part according to embodiments of the present disclosure;

FIG. 11 illustrate the subassembly in an initial state prior to rotating the proximal housing part according to embodiments of the present disclosure;

FIG. 12 illustrate the subassembly after rotating the proximal housing part according to embodiments of the present disclosure;

FIG. 13 is a perspective view of a medicament delivery device showing the activation interface and locking element according to embodiments of the present disclosure;

FIG. 14 is a perspective view of a medicament delivery device showing the activation interface and locking element according to embodiments of the present disclosure;

FIG. 15 is a perspective view of a medicament delivery device showing the activation interface and locking element prior to activation according to embodiments of the present disclosure; and

FIG. 16 is a perspective view of a medicament delivery device showing the activation interface and locking element subsequent to activation according to embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

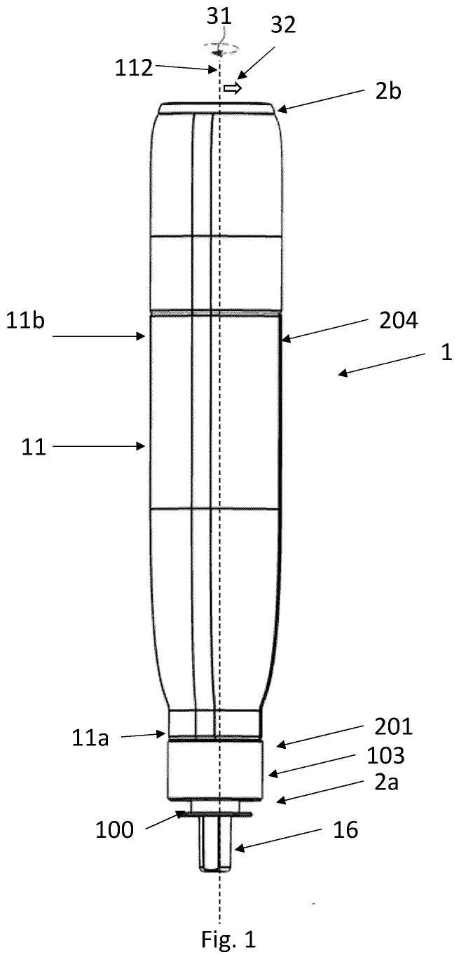

FIG. 1 shows an example of a medicament delivery device 1 such as an autoinjector according to embodiments of the present disclosure. The medicament delivery device 1 is configured to expel medicament from a medicament container via a medicament delivery member such as a needle, to a user at a dose delivery site. The medicament delivery device 1 extends from a proximal end 2a to a distal end 2b relative to the axis 112.

The medicament delivery device 1 comprises a housing 11 which has a proximal end 11a and a distal end 11b.

The medicament delivery device 1 further comprises a needle cover 100, and a removable needle shield 16 which may be provided as a so-called rigid needle shield.

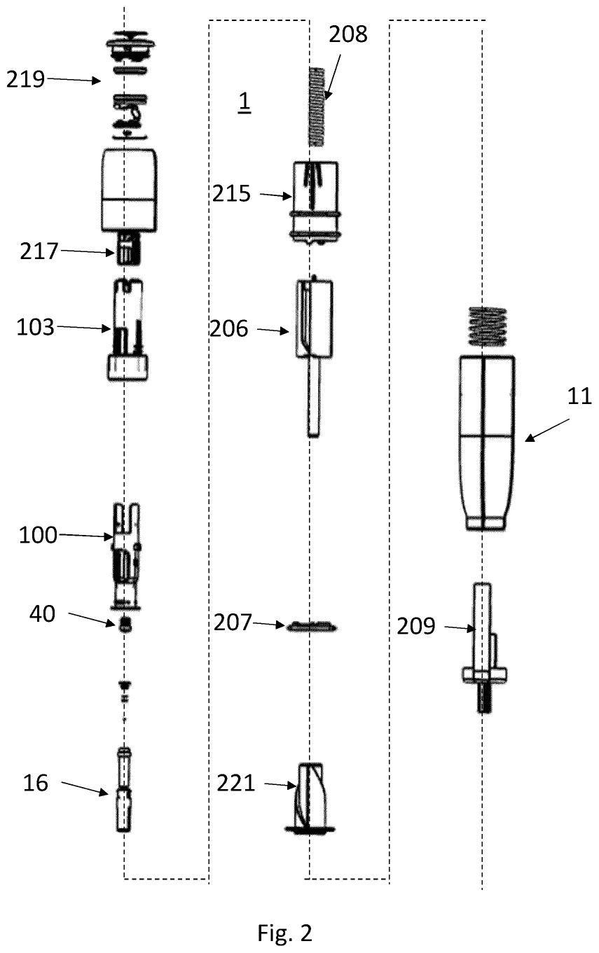

FIG. 2 is an exploded view of the medicament delivery device 1 which further comprises proximal housing part 103 removably attachable to the housing 11 of the medicament delivery device at a proximal end 11a of the housing 11.

The medicament delivery device 1 further comprises a plunger rod 206 configured to move a stopper 40 in the proximal direction upon being moved by the plunger rod spring 208, so that a medicament contained in the medicament container is expelled.

The medicament delivery device 1 further comprises a locking element 207 configured to prevent the plunger rod 206 from moving axially in relation to the housing 11 in a locking position. An activation interface 209 that is movable in the housing 11 may release the locking element 207 to move out from the locking position. A trigger of the needle cover 100 contacts the locking element 207 via the activation interface 209 to release the plunger rod 206.

The medicament delivery device 1 further comprises a rotary spring support 215 for the spring 208, a rotator 217, an electronics unit 219, and a loading ramp 221.

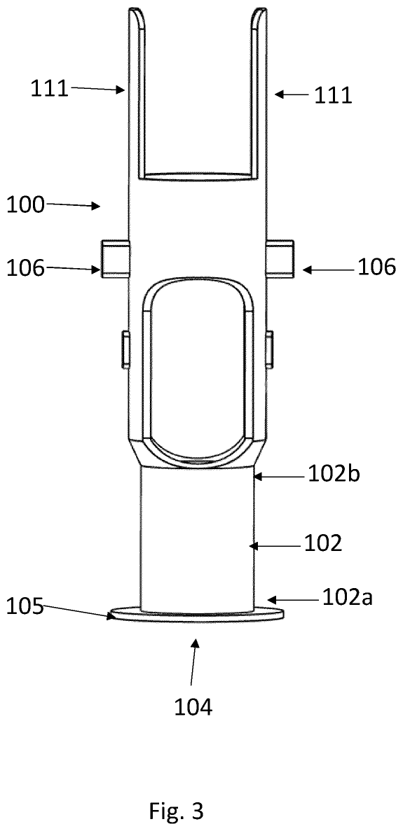

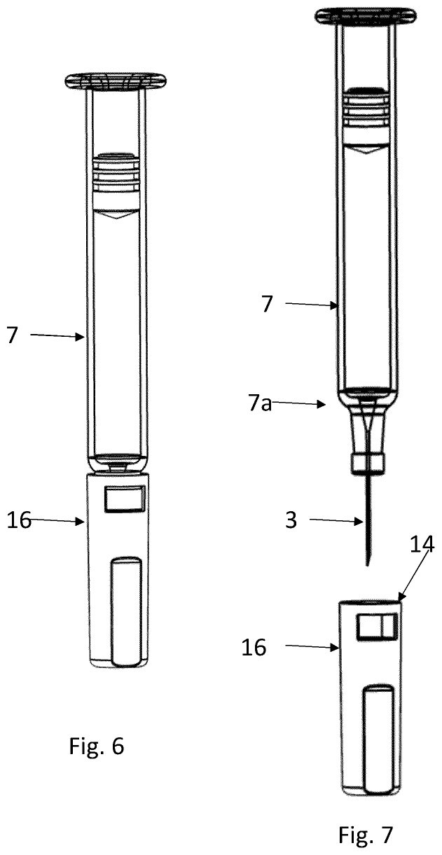

FIG. 3 is a perspective view of a needle cover 100 according to embodiments of the present disclosure. The needle cover 100 configured to cover a needle 3 attached to a proximal end 7a of a medicament container 7 which are better seen in FIGS. 6 and 7.

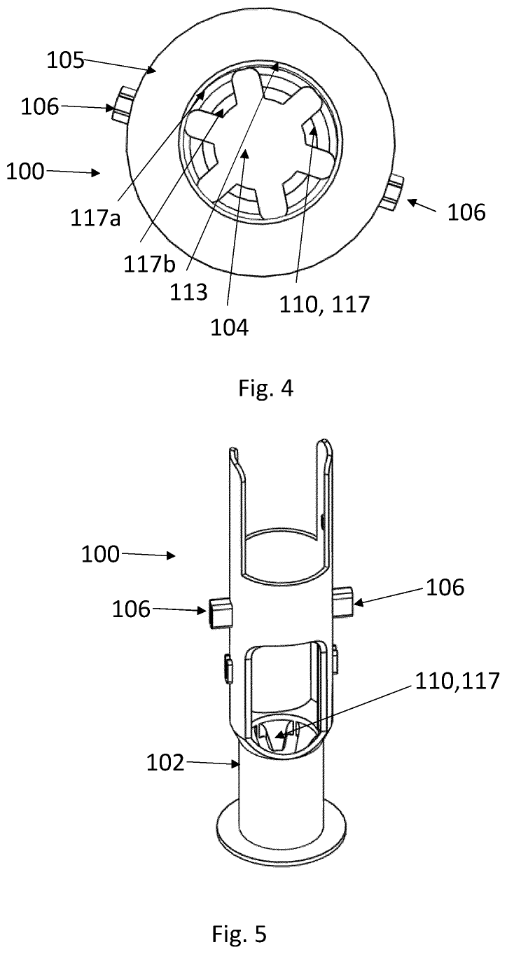

The needle cover 100 comprises a proximal tubular portion 102 defining a proximal opening 104 through which a needle 3 extends in a proximal direction during an injection event. FIG. 4 illustrates the needle cover 100 as seen from the proximal side and shows the opening 104 surrounded by the flange 105. The flange 105 is attached to the proximal tubular portion 102 at its proximal end 102a and is configured to come in contact with a user's skin during an injection event. The flange 105 is here shown with a circular shape which is the preferred shape. However, the flange can be any other suitable shapes. The flange is wider in the radial direction 32 than the proximal tubular portion 102.

Further, at least one protrusion 106 extends radially outwards. In this particular embodiment, there are two protrusions 106 that extend radially outwards. The two protrusions 106 are arranged on opposite sides of the needle cover 100 with respect to the longitudinal axis 102. The needle cover 100 further comprises arms 111 on the distal end of the needle cover 100. The arms 111 extend in the distal direction.

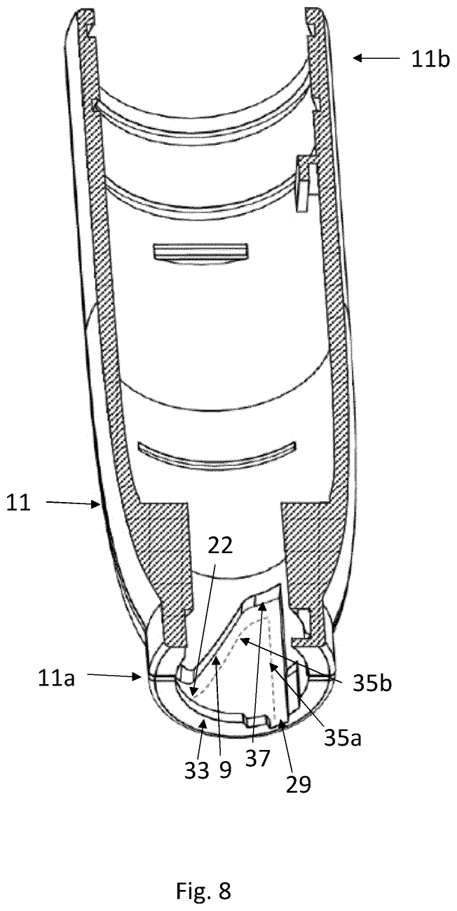

The protrusion 106 is configured to slide against a surface 9 of the housing 11 accommodating the needle cover 100. A cut view of the housing 11 is shown in FIG. 8 in which the surface 9 is shown arranged in a proximal end 11a of the housing 11. It is envisaged that any one of the protrusions 106 and the surface 9 of the housing includes a curved surface, however in this specific embodiment, the curved surface 9 is comprised in the housing 11. The interface between the curved surface 9 and the protrusion 16 is configured as a cam interface which is configured to cause the needle cover 100 to move proximally with respect to the housing 11 when the needle cover 100 is rotated axially relative the housing 11.

It is envisaged that the at least one protrusion 106 of the needle cover 100 extending radially outwards comprises the curved surface configured to slide against the surface 9 that is part of a protrusion of the housing 11.

Turning now to FIGS. 4 and 5, the needle cover further comprises at least one protrusion 110 arranged inside the proximal tubular portion 102. The at least one protrusion 110 extends radially inwards towards the longitudinal axis 112 of the needle cover 100. The at least one protrusion 110 is configured to engage with a distally facing surface 14 of a removable shield member 16, better seen in FIGS. 6 and 7 arranged to cover the needle. The engagement between the protrusion and the surface 14 pushes the removable shield member 16 in the proximal direction to remove the removable shield member 16 from the medicament container 7.

This protrusion 110 can either be in the form of a flange that extends radially inwards or as in the illustrated embodiment, in the form of a set of flexible tabs 117. In case of a flange, the flange is annular and extends along the full inner circumference of the proximal tubular portion 102 and is configured to abut against and push on a distally facing surface 14 of the removable shield member 16. In other words, the annular flange is preferably adjacent to the distally facing surface 14 of the removable shield member 16.

In the depicted embodiment, the at least one protrusion 110 is a set of flexible tabs 117 annularly arranged around an inner circumference 113 of the needle cover 100. In this case the set of flexible tabs 117 are configured to abut against and push on a distally facing surface 14 of the removable shield member 16. The flexible tabs 117 comprises a fixed end 117a attached to the tubular body of the needle cover 100. The flexible tabs 117 further comprises a free end 117b which is movable with respect to the fixed end 117a. The free end 117b abuts against and push on the distally facing surface 14 of the removable shield member 16. Thus, preferably, the flexible tabs 117, or at least the free ends 117b thereof, are adjacent to the distally facing surface 14 of the removable shield member 16.

Further, the at least one protrusion 110 extending radially inwards is arranged at the distal end 102b of the proximal tubular portion 102. Depending on the specific implementation it is envisaged that the protrusions 110 are arranged elsewhere, for example in a more proximal location of the proximal tubular portion 102.

The proximal tubular portion of the needle cover is configured to shield the needle 3 after an injection event is completed.

The needle cover is generally configured to be moved relative to the housing 3 from an extended position to a retracted position in which the needle cover 100 is received further in the housing 11 and in which the needle 3 is exposed.

The medicament delivery device 1 comprises the subassembly 2 which will now be described in more detail with reference to subsequent drawings.

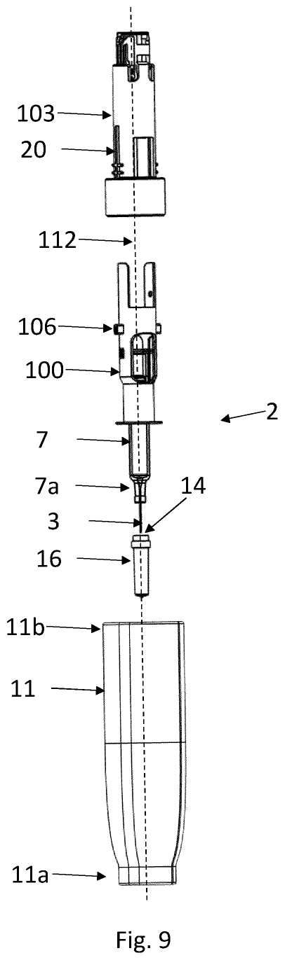

An exploded view of the subassembly 2 is shown in FIG. 9. The subassembly comprises the housing 11 extending in a longitudinal direction from a proximal end 11a to a distal end 11b relative the longitudinal axis 112.

The needle cover 100 arranged in the housing 11 and configured to cover a needle 3 at a proximal end 11a of the housing 11. The needle cover 100 is axially movable in relation to the housing 11 along a longitudinal axis 112 between a distal position and a proximal position.

The removable shield member 16 being configured to cover the needle 3 which is attached to a proximal end 7a of a medicament container 7 accommodated in the housing 11.

As described above, the needle cover comprising at least one protrusion 106 extending radially outwards and being configured to slide against a surface 9 of the housing 11. At least one of the protrusion 106 and the surface 9 of the housing 11 includes a curved surface to cause the needle cover 100 to move proximally with respect to the needle 3 when the needle cover 100 is rotated axially relative the housing.

The needle cover 100 further comprising at least one protrusion 110 as described in more detail above, extending radially inwards towards a longitudinal axis 112 of the needle cover. The least one protrusion 110 is adjacent to a distally facing surface 14 of the removable shield member 16. The subassembly further comprises a proximal housing part 103 removably attachable to the housing 11 of the medicament delivery device at a proximal end 11a of the housing.

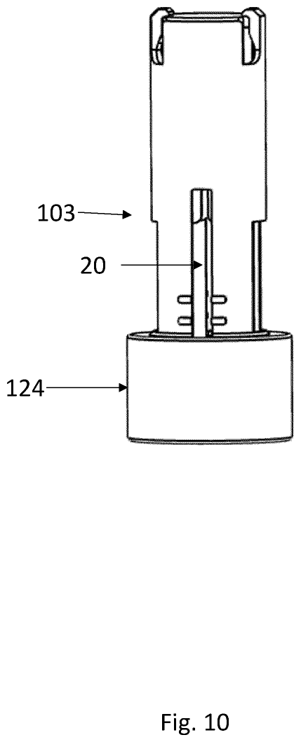

The proximal housing part 103 is illustrated in FIG. 10 and comprises a tubular proximal end 124 through which the flange 105 and the proximal tubular portion 102 of the needle cover 100 protrudes. The tubular proximal end 124 is here depicted with a diameter exceeding that of adjacent portions of the proximal housing part 103.

Further, the proximal housing part 103 comprises a slot 20 extending parallel with the longitudinal axis 112. The slot 20 is configured to receive the at least one protrusion 106 extending radially outwards of the needle cover.

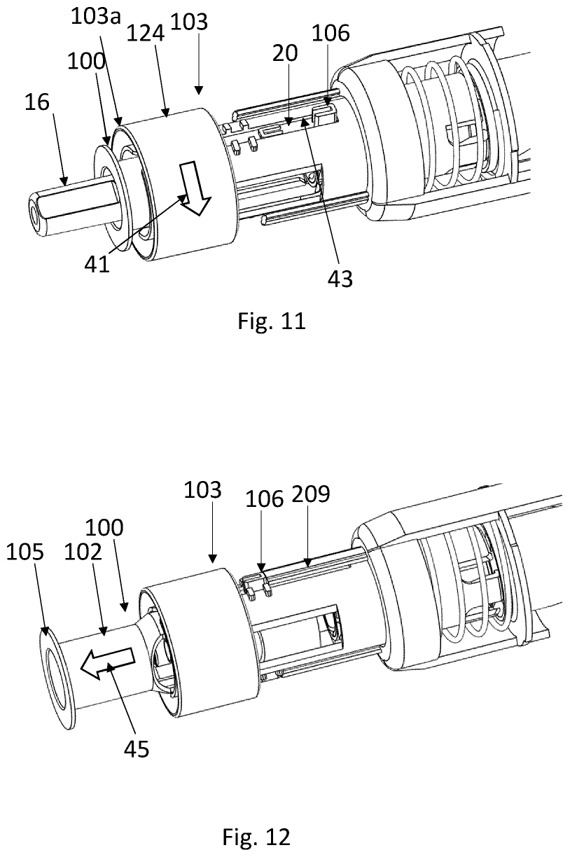

FIG. 11 illustrate the subassembly 2 in an initial state. Here, the proximal housing part 103 is received in the housing 11 (not shown). The protrusion 106 of the needle cover is arranged in the slot 20. The width of the slot 20 is slightly larger than the width of the protrusion 106 so that only little slack is allowed. However, the protrusion 106 is allowed to slide along the longitudinal extension of the length of the slot 20. Further, when the proximal housing part 103 accommodating the needle cover 100 is attached to the housing 11, the protrusion 16 is received in an opening 29 of a proximal inner edge flange 33 of the housing 11, better seen in FIG. 8. The protrusion slides along path 35a until it reaches a distal pocket or surface 37. In this initial position, the needle cover is still in a distal position in which the removable shield member 16 is exposed at the proximal end 103a of the proximal housing part 103. It is however, also envisaged that the removable shield member 16 is fully covered by the needle cover 100 in this initial position.

As the proximal housing part 103 is rotated as conceptually illustrated by arrow 41, the side wall 43 of the slot 20 push on the protrusion 16 to force the needle cover 100 to rotate. Turning again to FIG. 8, when the needle cover 100 rotates the protrusion 16 slides against the curved surface 9 of the housing 11 along path 35b. This causes the needle cover 100 to move in the proximal direction indicated by directional arrow 45 in FIG. 12. The interaction between the protrusion 16 and the curved surface 9 is adapted as a cam interface where the rotation of the proximal housing part causes a rotation of the needle cover and a linear proximal motion by the interaction between the protrusion 16 and the curved surface 9. Thus, the proximal motion of the needle cover 100 caused by the interaction with the surface of the housing moves the needle cover to a proximal position relative the distal position.

Finally, the protrusion 16 abuts against a distally facing surface 22 of the housing 11 to lock the proximal housing part 103 in a locked position. The protrusion 16 is locked between distally facing surface 22 of the edge 33 of the housing 11 and a proximal locking surface of an activation interface 209. The locking is achieved by configured as a bayonet connection.

When the needle cover moves in the proximal direction as indicated in FIG. 12, the protrusions 110, or flexible tabs, push on the removable needle shield 16 so that the needle shield 16 is removed from the subassembly 2.

Again, with reference to FIGS. 1 and 2, in one aspect of the present disclosure there is provided a medicament delivery device 1 comprising a proximal disposable part 201 arranged with a proximal housing 103 and a needle cover 100 telescopically arranged in relation to the proximal housing 103. The proximal disposable part 201 is configured to receive a medicament container 7.

A distal reusable part 204 is detachably arranged with the proximal disposable part 201, for example, by the bayonet coupling described above.

The needle cover 100 has a proximal position in which the needle cover 100 is extended in the proximal direction to cover the needle 3 of the medicament container 7, and a distal position in which the needle cover 100 is retracted in the distal direction to expose the needle 3.

The distal reusable part 204 comprises a plunger unit 206 and a plunger rod spring 208. The plunger unit 206 is configured to move a stopper 40 of the medicament container 7 in the proximal direction upon being moved by the plunger rod spring 208, so that a medicament contained in the medicament container 7 is expelled.

The distal reusable power pack 204 comprises the housing 11, the housing 11 being configured to receive the proximal disposable part 201.

The proximal disposable part 201 is configured to be rotated with respect to reusable part 204 for attachment with the reusable part 204. The rotation causes a rotation of the needle cover 100 axially relative the housing, wherein the proximal motion of the needle cover 100 causes the at least one protrusion 106 extending radially inwards to remove the removable shield 16 member from the medicament container 7.

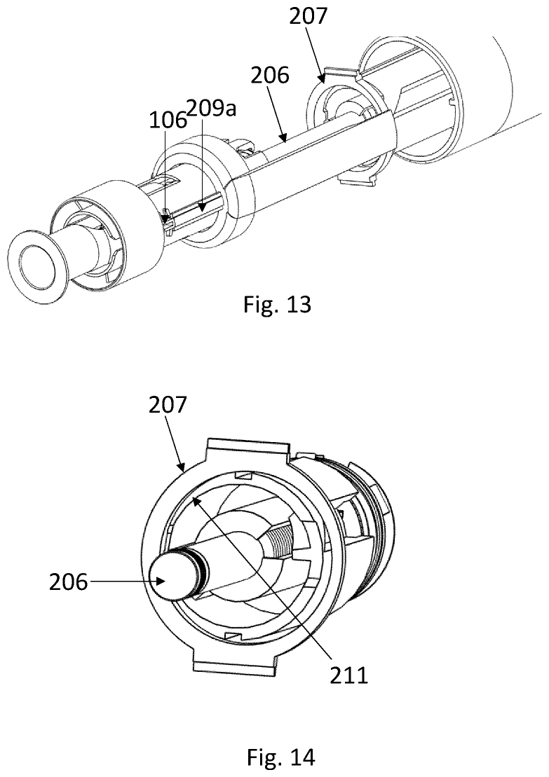

FIG. 13 illustrates the protrusion 106 of the needle cover 100 is configured as a trigger configured to release a locking element 207 to move out from the locking position, thereby releasing the plunger unit 206 to move in the proximal direction. As is better seen in FIG. 14, the locking element 207 is a locking ring which is off set with the plunger rod 206 so that the plunger rod is prevented from moving proximally. A distal surface of the locking ring 207 abuts against a proximal surface 211.

The medicament delivery device here comprising an activation interface 209 that is movable in the housing 11. The trigger 106 of the needle cover 100 contacts the locking element 207 via the activation interface 209 to release the locking element 207 to move out from the locking position. More specially, the activation interface 209 comprises an arm 209a that reaches to the trigger 106 when the needle cover has been rotated in the housing 11. The rotation of the needle cover 100 causes the trigger 106 to be is aligned with the activation interface 209, or more specifically with the arm 209a of the activation interface 209.

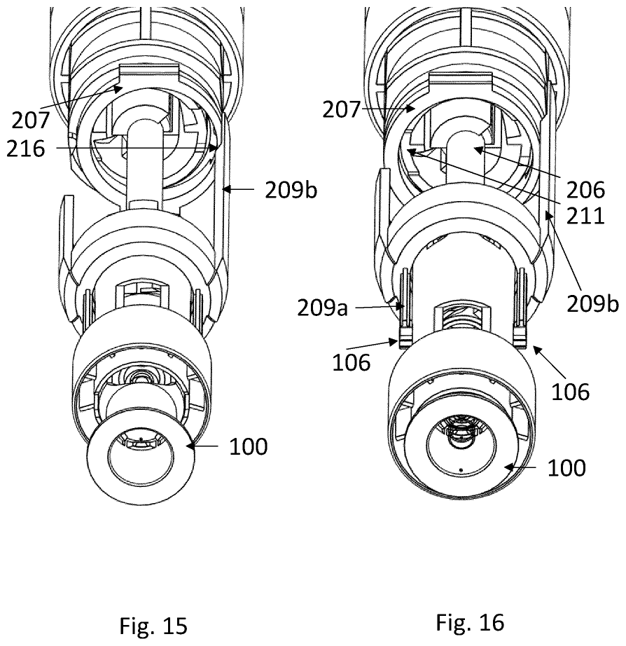

FIGS. 15 and 16 illustrate that the locking element 207 is a locking ring that is transversely movable in the housing to move out from the locking position. In FIG. 15 the needle cover 100 is in the proximal position where the trigger 106 has not yet pushed the activation interface 209 distally. The distally extending arm 209a of the activation interface 209 reaches towards the locking ring and comprises an inclined surface 216 facing radially inwards. When the needle cover is move towards its retracted, distal position shown in FIG. 16, the distally extending arm 209a of the activation interface 209 reaches the locking ring 209 so that the inclined surface 215 contacts the locking ring 207. The interaction between the inclined surface 216 and the locking ring move the locking ring transversely so that the locking ring move out from the locking position. Thus, in FIG. 16 the locking ring 207 and the plunger rod 206 are aligned so that the distal surface of the locking ring 207 no longer abuts against the proximal surface 211 of the plunger rod 206. In this position, the plunger rod can move proximally to move the stopper 40 of the medicament container 7 in the proximal direction to deliver an injection dose.

It is also conceivable that the activation interface 209 is excluded and the needle cover 100 comprises a distally extending arm configured to contact the locking element 207 to release the locking element to move out from the locking position. In this case, the rotation of the needle cover 100 causes the distally extending arm of the needle cover to be aligned with the locking element 207.

A medicament delivery device (such as an autoinjector) may generally include various other components. For example, a sensor unit which may recognize injection events, such as the autoinjector inserted into an attachment portion of e.g., a supporting pad, injection started, and injection ends, a memory unit which is configured to store the recorded data during the injection, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the supporting pad are configured to recognize the event and give feedback to the patient via haptic/visual or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer.

The mechanical switch, hall-effect sensor, or accelerometer can be used for detection of the insertion of the auto-injector into an injection port.

The accelerometer can be used for detecting injection events.

Possible wireless communication methods include Bluetooth and Cellular Networks.

Bluetooth connectivity requires a smart device to transmit the stored data to the network and it requires a pairing action between the support inpad and the smart device before being able to use the supporting pad in case of 2-way connection. But it's a cheaper alternative and it requires less space on PCB. A 1-way connection does not require pairing.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are described in the clauses below.

1. A needle cover (100) configured to cover a needle (3) of a medicament delivery device (1), the needle (3) being attached to a proximal end (7a) of a medicament container (7), the needle cover (100) comprising:
a proximal tubular portion (102) defining a proximal opening (104) through which a needle (3) extends in a proximal direction during an injection event,
at least one protrusion (106) extending radially outwards and being configured to slide against a surface (9) of a housing (11) accommodating the needle cover (100), wherein the at least one protrusion (106) and the surface of the housing includes a curved surface to cause the needle cover (100) to move proximally with respect to the housing (11) when the needle cover (100) is rotated axially relative the housing (11); and
at least one protrusion (110) arranged inside the proximal tubular portion (102), the at least one protrusion (110) extends radially inwards towards the longitudinal axis (112) of the needle cover, the at least one protrusion (110) extending radially inwards is configured to engage with a distally facing surface (14) of a removable shield member (16) arranged to cover the needle, and to push the removable shield member (16) in the proximal direction to remove the removable shield member from the medicament container (7).

2. The needle cover according to clause 1, the at least one protrusion extending radially inwards is an annular flange configured to abut against and push on a distally facing surface of the removable shield member (16).

3. The needle cover according to clause 1, at least one protrusion extending radially inwards is a set of flexible tabs (117) annularly arranged around an inner circumference (113) of the needle cover (100) and being configured to abut against and push on a distally facing surface (14) of the removable shield member (16).

4. The needle cover according to any one of the preceding clauses, wherein the proximal tubular portion (102) is configured to shield the needle (3) after an injection event is completed.

5. The needle cover according to any one of the preceding clauses, wherein the at least one protrusion (110) extending radially inwards is arranged at the distal (102b) end of the proximal tubular portion.

6. The needle cover according to any one of the preceding clauses, comprising two protrusions (106) extending radially outwards symmetrically arranged on the needle cover (100) on opposite sides of the longitudinal axis (112).

7. A subassembly (2) of a medicament delivery device (1), the subassembly comprising:
a housing (11) extending in a longitudinal direction from a proximal end (11a) to a distal end (11b) relative the longitudinal axis (112),
a needle cover (100) arranged in the housing (11) and configured to cover a needle (3) at a proximal end (11a) of the housing (11), the needle cover is axially movable in relation to the housing (11) along a longitudinal axis between a distal position and a proximal position; a removable shield member (16) configured to cover a needle (3), the needle (3) being attached to a proximal end (7a) of a medicament container (7) accommodated in the housing (11);
the needle cover comprising at least one protrusion (106) extending radially outwards and being configured to slide against a surface (9) of the housing (11), wherein at least one of the protrusions (106) and the surface (9) of the housing includes a curved surface to cause the needle cover to move proximally with respect to the needle (3) when the needle cover is rotated axially relative the housing; and
the needle cover further comprising at least one protrusion (110) extending radially inwards towards a longitudinal axis of the needle cover, the least one protrusion extending radially inwards is adjacent to a distally facing surface (14) of the removable shield member.

8. The subassembly according to clause 7, comprising a proximal housing part (103) removably attachable to the housing (11) of the medicament delivery device at a proximal end (11a) of the housing (11).

9. The subassembly (2) according to clause 7, wherein the proximal housing part comprises a slot (20) extending parallel with the longitudinal axis in which the at least one protrusion extending radially outwards of the needle cover is arranged.

10. The subassembly (2) according to any one of clauses 7 to 9, wherein the at least one protrusion extending radially outwards of the needle cover abuts against a distally facing surface (22) of the housing (11) to lock the proximal housing part (103) in a locked position.

11. The subassembly (2) according to any one of clauses 7 to 10, wherein the needle cover (100) is initially in a distal position in which the removable shield member (16) is exposed at the proximal end (103a) of the proximal housing part.

12. The subassembly (2) according to clause 11, wherein the proximal motion of the needle cover (100) caused by the interaction with the surface (9) of the housing moves the needle cover to a proximal position relative the distal position.

13. The subassembly according to any one of clauses 7 to 12, wherein the at least one protrusion extending radially inwards is an annular flange adjacent to the distally facing surface of the removable shield member.

14. The subassembly according to any one of clauses 7 to 12, the at least one protrusion extending radially inwards is a set of flexible tabs (117) annularly arranged around an inner circumference of the needle cover and being adjacent to the distally facing surface of the removable shield member.

15. The subassembly according to any one of clauses 7 to 14, wherein the surface (9) of the housing comprises the curved surface.

16. The subassembly according to any one of clauses 7 to 14, wherein the at least one protrusion (106) of the needle cover extending radially outwards comprises the curved surface configured to slide against the surface (9) that is part of a protrusion of the housing (11).

17. A medicament delivery device comprising the subassembly according to any one of clauses 7 to 16.

18. A medicament delivery device (1) comprising:
a proximal disposable part (201) arranged with a proximal housing (103) and a needle cover (100) telescopically arranged in relation to the proximal housing (103); wherein the proximal disposable part (201) is configured to receive a medicament container (7);
a distal reusable part (204) detachably arranged with the proximal disposable part (201);
wherein the needle cover (100) has an extension position in which the needle cover (100) is extended in the proximal direction to cover a needle (3) of the medicament container (7), and a distal position in which the needle cover (100) is retracted in the distal direction to expose the needle (3);
wherein the distal reusable part (204) comprises a plunger unit (206) and a plunger rod spring (208);
wherein the plunger unit (206) is configured to move a stopper of the medicament container (7) in the proximal direction upon being moved by the plunger rod spring (208), so that a medicament contained in the medicament container is expelled;
wherein the distal reusable power pack (204) comprises a housing (11), the housing (11) being configured to receive the proximal disposable part (201).

19. The medicament delivery device according to clause 18, further comprises a locking element (207) configured to prevent the plunger rod from moving axially in relation to the housing (11) in a locking position.

20. The medicament delivery device according to any one of clauses 18 and 19, wherein the needle cover (100) comprises a trigger; wherein the trigger (106) is configured to release the locking element (207) to move out from the locking position, thereby releasing the plunger unit (206) to move in the proximal direction.

21. The medicament delivery device according to clause 20, comprising an activation interface (209) that is movable in the housing, wherein the trigger of the needle cover contacts the locking element (209) via the activation interface to release the locking element (209) to move out from the locking position.

22. The medicament delivery device according to clause 20, wherein the needle cover comprises a distally extending arm configured to contact the locking element to release the locking element to move out from the locking position.

23. The medicament delivery device according to any one of clauses 19 to 22, wherein the locking element is a locking ring (209) that is transversely movable in the housing to move out from the locking position.

24. The medicament delivery device according to any one of clauses 18 to 23, wherein the needle cover comprises:
at least one protrusion (106) extending radially outwards and being configured to slide against a surface (9) of the housing (11) to cause the needle cover to move proximally with respect to the needle (3) when the needle cover is rotated axially relative the housing once received in the housing (11); and
at least one protrusion (110) extending radially inwards towards a longitudinal axis of the needle cover, the least one protrusion extending radially inwards is adjacent to a distally facing surface (14) of a removable shield member covering the needle.

25. The medicament delivery device according to clause 24, wherein the proximal disposable part (201) is configured to be rotated with respect to distal reusable part (204) for attachment with the distal reusable part whereby the rotation causes the rotation of the needle cover (100) axially relative the housing (11), wherein the proximal motion of the needle cover causes the at least one protrusion extending radially inwards to remove the removable shield member from the medicament container.

26. The medicament delivery device according to clause 21 and clause 25, wherein the rotation of the needle cover causes the trigger to be is aligned with the activation interface (209).

27. The medicament delivery device according to clause 25 and clause 22, wherein the rotation of the needle cover causes the distally extending arm of the needle cover to be is aligned with the locking element.

28. The medicament delivery device according to any one of clauses 24 to 25, wherein the surface (9) of the housing comprises the curved surface.

29. The medicament delivery device according to any one of clause 24 to 25, wherein the at least one protrusion (106) of the needle cover extending radially outwards comprises the curved surface configured to slide against the surface (9) that is part of a protrusion of the housing (11).

30. The medicament delivery device according to any one of clauses 18 to 29, wherein the proximal housing part comprises a slot (20) extending parallel with the longitudinal axis in which the at least one protrusion extending radially outwards of the needle cover is arranged.

31. The medicament delivery device according to any one of clauses 18 to 30, wherein the at least one protrusion (106) extending radially outwards of the needle cover abuts against a distally facing surface (22) of the housing (11) to lock the proximal housing part in a locked position.

32. The medicament delivery device according to any one of clauses 18 to 31, wherein the needle cover (100) is initially in a distal position in which the removable shield member (16) is exposed at the proximal end (103a) of the proximal housing part (103).

33. The medicament delivery device according to clause 32, wherein the proximal motion of the needle cover (100) caused by the interaction with the surface of the housing moves the needle cover to a proximal position relative the distal position.

34. The medicament delivery device according to any one of clauses 18 to 33, wherein the at least one protrusion (110) extending radially inwards is an annular flange adjacent to the distally facing surface of the removable shield member.

35. The medicament delivery device according to any one of clauses 18 to 33, the at least one protrusion extending radially inwards is a set of flexible tabs (117) annularly arranged around an inner circumference of the needle cover and being adjacent to the distally facing surface of the removable shield member.

1-15. (canceled)

16. A medicament delivery device comprising:
a proximal disposable part arranged with a proximal housing and a needle cover telescopically arranged in relation to the proximal housing, wherein the proximal disposable part is configured to receive a medicament container; and
a distal reusable part detachably arranged with the proximal disposable part,
wherein the needle cover has an extension position in which the needle cover is extended in the proximal direction to cover a needle of the medicament container, and a distal position in which the needle cover is retracted in the distal direction to expose the needle,
wherein the distal reusable part comprises a plunger unit and a plunger rod spring,
wherein the plunger unit is configured to move a stopper of the medicament container in the proximal direction upon being moved by the plunger rod spring, so that a medicament contained in the medicament container is expelled,
wherein the distal reusable power pack comprises a housing, the housing being configured to receive the proximal disposable part.

17. The medicament delivery device according to claim 16, further comprises a locking element configured to prevent the plunger rod from moving axially in relation to the housing in a locking position.

18. The medicament delivery device according to claim 16, wherein the needle cover comprises a trigger, and wherein the trigger is configured to release the locking element to move out from the locking position, thereby releasing the plunger unit to move in the proximal direction.

19. The medicament delivery device according to claim 18, comprising an activation interface that is movable in the housing, wherein the trigger of the needle cover contacts the locking element via the activation interface to release the locking element to move out from the locking position.

20. The medicament delivery device according to claim 18, wherein the needle cover comprises a distally extending arm configured to contact the locking element to release the locking element to move out from the locking position.

21. The medicament delivery device according to claim 17, wherein the locking element is a locking ring that is transversely movable in the housing to move out from the locking position.

22. The medicament delivery device according to claim 16, wherein the needle cover comprises:
at least one protrusion extending radially outwards and being configured to slide against a surface of the housing to cause the needle cover to move proximally with respect to the needle when the needle cover is rotated axially relative the housing once received in the housing; and
at least one protrusion extending radially inwards towards a longitudinal axis of the needle cover, the least one protrusion extending radially inwards is adjacent to a distally facing surface of a removable shield member covering the needle.

23. The medicament delivery device according to claim 22, wherein the proximal disposable part is configured to be rotated with respect to distal reusable part for attachment with the distal reusable part whereby the rotation causes the rotation of the needle cover axially relative the housing, wherein the proximal motion of the needle cover causes the at least one protrusion extending radially inwards to remove the removable shield member from the medicament container.

24. The medicament delivery device according to claim 23, wherein the rotation of the needle cover causes the trigger to be is aligned with the activation interface.

25. The medicament delivery device according to claim 23, wherein the rotation of the needle cover causes the distally extending arm of the needle cover to be is aligned with the locking element.

26. The medicament delivery device according to claim 22, wherein the surface of the housing comprises the curved surface.

27. The medicament delivery device according to claim 22, wherein the at least one protrusion of the needle cover extending radially outwards comprises the curved surface configured to slide against the surface that is part of a protrusion of the housing.

28. The medicament delivery device according to claim 16, wherein the proximal housing part comprises a slot extending parallel with the longitudinal axis in which the at least one protrusion extending radially outwards of the needle cover is arranged.

29. The medicament delivery device according to claim 16, wherein the at least one protrusion extending radially outwards of the needle cover abuts against a distally facing surface of the housing to lock the proximal housing part in a locked position.

30. The medicament delivery device according to claim 16, wherein the at least one protrusion extending radially inwards is an annular flange adjacent to the distally facing surface of the removable shield member.
